(19) United States
(12) Patent Application Publication
(10) Pub. No.: US 2026/0258218 A1
(43) Pub. Date: Sep. 3, 2026

(54) PROCESS FOR THE DEPOLYMERIZATION OF A POLYSTYRENE FEEDSTOCK BY PYROLYSIS

(71) Applicant: COMPAGNIE GENERALE DES ETABLISSEMENTS MICHELIN, Clermont-Ferrand (FR)

(72) Inventors: LISE MAISONNEUVE, Clermont-Ferrand (FR); FLORENT MATHIEU, Clermont-Ferrand (FR)

(21) Appl. No.: 18/875,459

(22) PCT Filed: Jun. 27, 2023
(86) PCT No.: PCT/EP2023/067414

§ 371 Date: Dec. 16, 2024

(30) Foreign Application Priority Data
Jun. 30, 2022 (FR) 2206627

(51) Int. Cl. C08J 11/12; C08J 11/08

(52) U.S. Cl. CPC C08J 11/12; C08J 11/08; C08J 2325/06

(57) ABSTRACT

A process for depolymerizing a polystyrene feedstock comprises at least two pyrolysis sections, in which the hydrogen partial pressure and/or the residence time of the gas phase are controlled in the pyrolysis section in step d).

### TECHNICAL FIELD OF THE INVENTION

The present invention relates to the field of processes for depolymerizing polystyrene compounds, in particular polystyrene, with a view to producing at least one styrene monomer.

### PRIOR ART

Styrene is a monomer very widely used in industry, whether for example for the production of polystyrene, which has multiple fields of application, or for the production of elastomers, such as styrene-butadiene rubber (SBR). It can be obtained in many ways, the main one being by the dehydrogenation of ethylbenzene, or to a lesser extent by the oxidation of ethylbenzene, followed by reaction with propylene and then dehydration of the product obtained.

With a view to reducing the pressure on fossil resources, a focus of recent developments has been the depolymerization of styrene compounds such as polystyrene. Such a process is described for example in patent application US 2021/0277202.

During these processes, polystyrene is decomposed at high temperature in an anaerobic environment into lower-molecular-weight compounds, including styrene. However, significant amounts of other products are also generated, in particular styrene oligomers. These oligomers can represent up to 40% by weight of the polystyrene feed and comprise predominantly styrene dimers and trimers, including in particular 1,3-diphenylpropane, 1,3-diphenyl-1-butene, 1,2-diphenylpropane, 1,3-diphenylbutane and 1,4-diphenylbenzene. The presence of these oligomers can be explained firstly by partial depolymerization reactions of polystyrene to oligomers and secondly by the polymerization or free-radical recombination of styrene or of oligomers in the depolymerization reactor and/or in the streams exiting said reactor. Document US 2021/0277202 proposes steam cracking the styrene oligomers produced in order to generate lighter compounds such as ethene, propene or benzene.

Other routes can be envisaged for exploiting the styrene oligomers and improving the overall styrene yield of these processes. Document U.S. Pat. No. 10,731,080 proposes recycling these oligomers into the feedstock of the pyrolysis reactor. However, this recycling approach, which is typically employed in processes with incomplete conversion into the product of interest, is not entirely satisfactory in this instance.

This is because at ambient pressure the oligomers have a boiling point close to the decomposition temperature of polystyrene. The recycling of the oligomers to the depolymerization reactor affects the chemistry of the decomposition of polystyrene by reducing the monomer selectivity and yield, since the vaporization of the oligomers will be in competition with the decomposition of the polystyrene. By consuming a significant proportion of the energy, the vaporization of styrene oligomers thus reduces the amount of energy available for polystyrene decomposition, thereby affecting the reaction temperature, reaction yield and selectivity in respect of styrene monomer production. This solution is therefore capable of further improvement.

Another solution with the aim of countering the effects presented above could consist of increasing the operating pressure of the pyrolysis section in order to increase the vaporization temperature of the styrene oligomers and the degree of vaporization at the operating temperature of the pyrolysis. However, this increase in operating pressure will also increase the vaporization temperature of the styrene, increasing its residence time in the liquid and gas phase, where it will decompose further into lighter compounds such as lighter non-condensable products (hydrogen and also C1, C3, C4 and C5 alkanes and alkenes). All in all, the increase in reactor pressure decreases the yield of liquid produced and the yield of styrene.

Document WO 2021/180893 proposes a process in which a polystyrene feedstock is pyrolyzed in a first reactor operated at a pressure of less than 1 bar and then separated into a light fraction comprising at least a portion of the styrene monomer and a heavy fraction comprising at least a portion of the styrene oligomers, this heavy fraction being treated in a second pyrolysis reactor operated under conditions different from the first pyrolysis reactor, in particular a pressure of greater than 1 bar and a temperature of less than 650° C. However, control of the composition of the heavy fraction is not addressed, but does have a major influence on the performance of the second pyrolysis reactor.

In the pursuit of its research activities, the applicant has discovered a process for depolymerizing a polystyrene feedstock in which the conversion of the polystyrene feedstock into styrene monomer is improved by employing two pyrolysis steps and through the control of the performance of the second step, and in particular by regulating the presence of a hydrogen donor.

### DETAILED DESCRIPTION OF THE INVENTION

The invention thus relates to a process for depolymerizing a polystyrene feedstock, comprising at least the following steps:
a. a step of preparing the polystyrene feedstock so as to bring about the melting of the plastic compounds,
b. a step of performing a first pyrolysis of the feedstock resulting from step a) involving a pyrolysis section and producing at least a first gaseous pyrolysis stream and a first liquid pyrolysis stream,
c. a separating step supplied at least by the first gaseous pyrolysis stream from step b) and the second gaseous pyrolysis stream from step d) and producing at least a stream rich in light compounds, a stream rich in oligomers, and a stream rich in aromatics,
d. a step of performing a second pyrolysis involving a pyrolysis section supplied at least by the stream rich in oligomers from step c) and producing at least a second gaseous pyrolysis stream and a second liquid pyrolysis stream,
e. a step of separating the stream rich in aromatics from step c) into at least a stream comprising predominantly ethylbenzene, a stream comprising predominantly styrene and a stream of heavy compounds,
in which the hydrogen partial pressure and/or the residence time of the gas phase are controlled in the pyrolysis section in step d).

### Definitions

The carbon-containing compounds mentioned in the description may be fossil in origin or be biobased. In the latter case, they may be partially or completely derived from biomass or obtained from renewable raw materials derived from biomass. This concerns in particular polymers, plasticizers, fillers, etc.

Any interval of values denoted by the expression “between a and b” represents the range of values extending from more than a to less than b (i.e. excluding the limits a and b), whereas any interval of values denoted by the expression “from a to b” means the range of values extending from a to b (i.e. including the strict limits a and b).

The term “hydrogen donor” denotes a compound capable of reacting with styrene oligomers during the pyrolysis reaction to provide hydrogen atoms. Such a compound may be for example a wax such as a paraffin wax, a thermoplastic polyolefin such as polyethylene or polypropylene, or dihydrogen.

“Paraffin wax” is understood as meaning, in a known manner, a linear or branched alkane that is solid at ambient temperature and has a melting point of less than 100° C. A paraffin wax typically contains from 20 to 40 carbon atoms.

“Styrene monomer” is understood as meaning the compound styrene, of formula C8H8.

The term “predominant compound” is understood as meaning that the compound is present to an extent of more than 50% by weight in the stream concerned.

### Feedstock of the Depolymerization Process

The process of the invention is a process for depolymerizing a polystyrene feedstock. A polystyrene feedstock is understood as meaning a feedstock that comprises styrene-based polymers, such as styrene rubbers and polystyrene.

The feedstock of the process is preferably a polystyrene feedstock resulting from plastic waste. Such a feedstock preferably comprises at least 90% by weight of polystyrene, preferably at least 93% by weight of polystyrene, and more preferably at least 95% by weight of polystyrene. The polystyrene feedstock may contain other compounds, particularly if it is derived from plastic waste. These other compounds may include, but are not limited to, plastic compounds such as polyethylene, polypropylene, elastomers, organic materials such as paper, food, or inorganic materials such as glass, metal or sand.

The polystyrene feedstock of the process of the invention may contain a hydrogen donor within the meaning of the present invention. For example, where the hydrogen donor is polypropylene, the polypropylene to polystyrene weight ratio in the polystyrene feedstock can be adjusted to between 1% by weight and 15% by weight, preferably between 1% and 3% by weight.

Step a)—Preparation

The process of the invention comprises a step of preparing the polystyrene feedstock. During this step, the polystyrene feedstock is conditioned so as to be able to supply the first pyrolysis step b). This preparation step may comprise shredding, degassing and heating operations in order to bring about melting of the plastic compounds, for example in an extrusion device in which the temperature is gradually increased. The vapour effluents (water, light compounds generated by the partial decomposition of the polystyrene feedstock) and solid effluents (non-meltable debris such as metal debris, glass debris) are separated.

The polystyrene feedstock is preferably heated gradually to a temperature of between 200° C. and 300° C., this temperature making it possible to achieve melting of the polystyrene while limiting its thermal decomposition.

Step b)—First Pyrolysis

The process of the invention comprises a step of a first pyrolysis of the feedstock resulting from step a) involving a pyrolysis section and producing at least a first gaseous pyrolysis stream and a first liquid pyrolysis stream.

Pyrolysis is understood as meaning the thermal decomposition of compounds in an inert atmosphere.

The conditioned feedstock resulting from step a), preferably in molten form, supplies a pyrolysis section operated at a temperature and a pressure such that the depolymerization of the polystyrene into styrene oligomers and into styrene monomer takes place. Preferably, the pyrolysis section is operated at a temperature ranging from 300 to 700° C., preferably ranging from 300° C. to 600° C. Preferably, the pyrolysis section is operated at a pressure ranging from 0.1 bar to 2 bar, preferably ranging from 0.5 bar to 1.5 bar and very preferably from 0.8 bar to 1.2 bar. Under these conditions, the amount of styrene oligomers in the first gaseous pyrolysis stream may be up to 40% by weight relative to the amount of polystyrene in the pyrolysis section feedstock.

The pyrolysis section preferably employs a microwave pyrolysis section. Such a microwave pyrolysis employable for the pyrolysis of a polystyrene feedstock is described for example in document WO 2020/202089.

The use of a microwave-assisted pyrolysis section allows higher heat transfer rates and higher reaction temperatures to be achieved, which favour end-of-chain scission reactions and minimize the formation of styrene oligomers. A microwave pyrolysis section is also characterized by a temperature in the reaction mass lower than that of a conventional pyrolysis section. The lowering of the temperatures in the reaction mass results in lower levels of vaporization of the styrene oligomers and avoids “overcracking” the styrene produced. The use of a microwave pyrolysis section will reduce the formation of styrene oligomers relative to a conventional pyrolysis section, but will not prevent them from forming altogether and in the first gaseous pyrolysis stream they may still represent up to 40% by weight relative to the amount of polystyrene in the pyrolysis section feedstock.

The pyrolysis section produces a first gaseous pyrolysis stream and a first liquid pyrolysis stream. The first gaseous pyrolysis stream may also contain droplets of entrained liquid. In addition to styrene oligomers, the first gaseous pyrolysis stream comprises the majority of the styrene monomer produced in the pyrolysis section, as well as light aromatic compounds that are gaseous under the operating conditions, such as alpha-methylstyrene, ethylbenzene, cumene and toluene.

The first gaseous pyrolysis stream comprises predominantly styrene, that is to say at least 50% by weight of styrene, preferably at least 60% by weight of styrene.

Preferably, the first gaseous pyrolysis stream contains not more than 10% by weight of ethylbenzene, preferably not more than 5% by weight of ethylbenzene and more preferably not more than 3% by weight of ethylbenzene.

The first gaseous pyrolysis stream preferably contains at least 10% by weight of compounds having a boiling point higher than that of styrene.

The first liquid pyrolysis stream may also comprise solid elements, unmelted polymers produced during the pyrolysis, or debris that are not separated in the feedstock preparation step. This stream preferably supplies a separation section in which any solid fraction is separated from the liquid fraction, it being possible for the latter to be recycled in a mixture with the material supplied to the pyrolysis section in the first pyrolysis step.

Step c)—Separation

The process of the invention comprises a separation step supplied at least by the first gaseous pyrolysis stream from step b) and the second gaseous pyrolysis stream from step d) and producing at least a stream rich in light compounds, a stream rich in oligomers, and a stream rich in aromatics.

The stream rich in light compounds comprises predominantly compounds lighter than benzene, in particular the compounds hydrogen, methane, ethane, ethylene, propane, propylene, butane, butene and isobutane.

The stream rich in aromatics comprises predominantly aromatic compounds containing from 6 to 9 carbon atoms. The separation step c) is operated such that 99% by weight of the styrene supplying this step is recovered in the stream rich in aromatics.

The stream rich in oligomers comprises predominantly styrene oligomers. The separation step c) is operated such that the stream rich in oligomers comprises less than 5% by weight of aromatic compounds containing from 6 to 9 carbon atoms. As well as maximizing the amount of aromatic compounds recovered in the stream rich in aromatics, minimizing the content of aromatic compounds containing from 6 to 9 carbon atoms in the stream rich in oligomers makes it possible to improve the functioning of the pyrolysis section in the second pyrolysis step d) while minimizing the proportion of the energy absorbed by the vaporization of these aromatic compounds in the pyrolysis section, vaporization that can cause problems with frothing and consequently adversely affect the correct functioning of this section.

The separation step c) is preferably carried out in a distillation column.

In this arrangement, the vapour effluent at the head of the column is cooled to a temperature of between 30° C. and 50° C., preferably between 35° C. and 45° C. The condensed liquid fraction is returned at the head of the column as reflux, while the vapour fraction is subsequently subcooled to a temperature of between −5° C. and 10° C., preferably between −5° C. and 5° C., so as to condense any styrene entrained with the light compounds. The stream condensed after subcooling is returned at the head of the column as reflux. The residual vapour fraction constitutes the stream rich in light compounds. This stream can subsequently be exploited, for example in the form of energy. A first cooling permits maximum use of cooling water at ambient temperature as a cold utility and minimizes the use of specific cold utility to achieve subcooling, which impacts favourably on the lifecycle analysis of the process of the invention.

In this arrangement, the distillation column is operated at a pressure of between 0.1 and 2.0 bara, preferably between 0.5 and 1.5 bara and more preferably between 0.5 and 1.1 bar, the operating pressure being understood as meaning the pressure measured at the head of the column. The term “bara” is understood as meaning bar absolute, as opposed to a pressure expressed in bar relative, commonly denoted “barg” according to the notation “bar gauge”.

In this arrangement, the distillation column is preferably supplied at the bottom of the column with at least the first gaseous pyrolysis stream from step b) and the second gaseous pyrolysis stream from step d) and produces at the head of the column a stream rich in light compounds and at the bottom a stream rich in oligomers and, through a sidestream withdrawal, a stream rich in aromatics, said column having as sole heat input said first gaseous pyrolysis stream from step b) and said second gaseous pyrolysis stream from step d).

The first gaseous pyrolysis stream from step b) and the second gaseous pyrolysis stream from step d) are at high temperature, preferably at a temperature higher than 300° C. This temperature is sufficient for the column not to require any other heat input.

By supplying the feedstock at the bottom of the column, the feedstock is rapidly cooled, thus making it possible to limit potential polymerization reactions of the styrene. This form of supply also permits better management of the “heavy” compounds. This is because the absence of a recirculation system at the bottom of the column, as is generally employed for maintaining the temperature of the distillation column, greatly limits the risk of fouling by the “heavy” compounds, which are particularly viscous.

The distillation column employed in step c) of the process of the invention comprises from 5 to 20 theoretical stages, preferably not more than 15 theoretical stages, more preferably from 8 to 12 theoretical stages.

A stream rich in aromatics is withdrawn at an intermediate plate. This withdrawal plate is located in the lower third of the distillation column, preferably 1 to 3 theoretical stages from the bottom plate. This withdrawal at a low position in the column, slightly away from the bottom plate, makes it possible to limit the entrainment of heavy compounds in the stream rich in aromatics and accordingly limits the risk of fouling of subsequent items of equipment.

In order to limit the risk of polymerization still further, an inhibitor of the polymerization of styrene to give polystyrene, such as 2,2,6,6-tetramethyl-4-oxopiperidinooxy, may preferably be supplied to the distillation column in step c) of the process, preferably at the head of the column.

Step d)—Second Pyrolysis

The process of the invention comprises a step of a second pyrolysis involving a pyrolysis section supplied at least by the stream rich in oligomers from step c) and producing at least a second gaseous pyrolysis stream and a second liquid pyrolysis stream.

In this step, the styrene oligomers are converted into monoaromatic compounds, in particular into styrene monomer. The pyrolysis section is operated at a temperature ranging from 400° C. to 900° C., preferably from 500° C. to 800° C., more preferably between 650° C. and 800° C. If the temperature is lower than 400° C., the liquid yield and the yield of monoaromatic compounds are inadequate, while above 900° C. these yields decline. The temperature range of between 650° C. and 800° C. makes it possible under the conditions of the invention to maximize both the liquid yield and the yield of monoaromatic compounds. The “liquid yield” is understood as meaning the percentage by weight of liquid obtained after pyrolysis of a given weight of stream rich in oligomers. The “yield of monoaromatic compounds” is understood as meaning the percentage by weight of monoaromatic compounds such as styrene, ethylbenzene, toluene, cumene or alpha-methylstyrene in the liquid fraction obtained after pyrolysis of a given weight of stream rich in oligomers.

The pyrolysis section is operated at a pressure ranging from 1.0 bar to 7.5 bar, preferably ranging from 3 bar to 6 bar and more preferably ranging from 3 bar to 4.5 bar. Increasing the operating pressure makes it possible to improve the yield of monoaromatic compounds by increasing the vapour pressure of the various constituents, but causes a decline in liquid yield. This pressure range makes it possible under the conditions of the invention to maximize both the liquid yield and the yield of monoaromatic compounds.

The pyrolysis section is preferably operated with a gas-phase residence time ranging from 10 s to 30 s, preferably ranging from 10 s to 25 s and very preferably ranging from 15 s to 20 s, the residence time being defined as the ratio of the volume (in m³) of the reactor (or reactors, if there are more than one in series) of the pyrolysis section and of the lines conveying the second gaseous pyrolysis stream until the separation step c) to the flow volume (in m³/s) of the second gaseous pyrolysis stream. This preferred residence time range makes it possible to maximize the yield of monoaromatic compounds. Below 10 s, the residence time is too short to permit adequate conversion, while beyond 30 s a possible recombination of monoaromatic compounds causes a decline in yield. The yield of monoaromatic compounds is maximized in particular for a residence time ranging from 15 s to 20 s. The residence time of the gas phase can be adjusted for example by supplying an inert gas to the pyrolysis section in step d), for example a gas selected from nitrogen, argon, helium, neon, xenon and krypton, preferably nitrogen.

The applicant has discovered that the yield of monoaromatic compounds, in particular of styrene monomer, can be controlled by adjusting the hydrogen partial pressure in the pyrolysis section in step d) and/or by controlling the residence time of the gas phase. This partial pressure is controlled by adjusting the hydrogen donor content in the polystyrene feedstock and/or in the supply to the pyrolysis section in step d).

The hydrogen partial pressure in the pyrolysis section in step d) may be increased by increasing the hydrogen donor content in the polystyrene feedstock and/or in the supply to the pyrolysis section in step d), or decreased for example by supplying an inert gas to the pyrolysis section in step d), for example a gas selected from nitrogen, argon, helium, neon, xenon and krypton, preferably nitrogen. The hydrogen donor content may be increased by mixing one or more hydrogen donors with the polystyrene feedstock and/or with the supply to the pyrolysis section in step d), it being possible that the polystyrene feedstock and/or the supply to the pyrolysis section in step d) already contains one or more hydrogen donors.

Thus, in one arrangement, the hydrogen partial pressure in the pyrolysis section in step d) may be adjusted by the presence of a hydrogen donor selected from polyolefins, paraffin waxes and mixtures thereof in the polystyrene feedstock and/or in the stream rich in oligomers from step c).

In another arrangement, optionally combined with the preceding arrangement, the hydrogen partial pressure may also be adjusted by the presence of a hydrogen donor selected from polyolefins, paraffin waxes, dihydrogen and mixtures thereof in the supply to the pyrolysis section in step d).

By adjusting the hydrogen donor content in the process of the invention and/or the residence time of the gas phase, it is thus possible to control the overall yield of monoaromatic compounds. The hydrogen donor content may be adjusted by measuring the content of monoaromatic compounds in the second gaseous pyrolysis stream; the optimum content may vary slightly depending on what is the desired result, for example to maximize the production of toluene and ethylbenzene, or of styrene.

For example, where the hydrogen donor is polypropylene, the polypropylene to polystyrene weight ratio in the polystyrene feedstock will be adjusted to between 1% by weight and 15% by weight, preferably between 1% and 3% by weight.

If the hydrogen donor content is too high, the yield of non-aromatic light products (hydrogen, methane, ethane, ethylene, propane, propylene, butane, butene, isobutane) and of solid residues resulting from the thermal degradation of compounds during pyrolysis increases, which lowers the overall yield of the products of interest.

The pyrolysis section preferably employs a microwave pyrolysis section. Such a microwave pyrolysis employable for the pyrolysis of a polystyrene feedstock is described for example in document WO 2020/202089.

Step e)—Separation

The process of the invention comprises a step of separating the stream rich in aromatics into at least a stream comprising predominantly ethylbenzene, a stream comprising predominantly styrene and a stream of heavy compounds.

The separation step e) makes it possible to obtain a stream comprising predominantly styrene that can be supplied to a process for the polymerization of styrene, thus meeting the specifications of such processes, having in particular a very high content of styrene, preferably of greater than 99.8% by weight, and very low contents of compounds, such as ethylbenzene, benzene, cumene, α-methylstyrene and styrene oligomers.

In a first preferred arrangement, step e) of separating the stream rich in aromatics comprises two successive separation sections.

A first separation section is supplied with the stream rich in aromatics from step c) and makes it possible to separate a stream comprising predominantly ethylbenzene and a styrene raffinate.

This first section is carried out in a distillation column comprising from 60 to 100 theoretical stages, and is operated at a pressure of less than or equal to 0.25 bara at the head of the column, so as to maintain the temperature at the column bottom at a value of less than or equal to 120° C.

The distillation column of the first section is supplied with the stream rich in aromatics from step c) at the bottom of the upper third of the column. For example, for a column comprising 60 theoretical stages, the stream rich in aromatics is supplied to a stage between the 18th theoretical stage and the 22nd theoretical stage, the stages being numbered from the top downwards.

The reflux ratio at the condenser of this column, corresponding to the reflux mass flow supplied at the head of the column to the mass flow of the stream comprising predominantly ethylbenzene, is preferably between 60 and 300. This parameter varies greatly, depending on the ethylbenzene content of the stream rich in aromatics. The lower the content of ethylbenzene in the stream rich in aromatics, the higher the reflux ratio at the condenser of the column.

The reflux ratio at the reboiler of this column, corresponding to the reflux mass flow supplied at the column bottom to the mass flow of styrene raffinate, is preferably between 4 and 10, more preferably between 5 and 9.

A second separation section is supplied with the styrene raffinate from the first separation section and produces a stream comprising predominantly styrene and a stream of heavy compounds.

This second section is carried out in a distillation column comprising from 40 to 100 theoretical stages, preferably comprising from 40 to 70 theoretical stages, and is operated at a pressure of less than or equal to 0.25 bara at the head of the column, so as to maintain the temperature at the column bottom at a value of less than or equal to 120° C.

The distillation column of the first section is supplied with the styrene raffinate from the first separation section in the lower portion of the column, preferably at the top of the bottom fifth of the column. For example, for a column comprising 50 theoretical stages, the styrene raffinate from the first separation section is supplied to a stage between the 35th theoretical stage and the 45th theoretical stage, the stages being numbered from the top downwards.

The reflux ratio at the condenser of this column, corresponding to the reflux mass flow supplied at the head of the column to the mass flow of the stream comprising predominantly styrene, is preferably between 4 and 8.

The reflux ratio at the reboiler of this column, corresponding to the reflux mass flow supplied at the column bottom to the mass flow of the stream of heavy compounds, is preferably between 40 and 200, this ratio being influenced greatly by the content of compounds such as cumene and α-methylstyrene.

In another preferred arrangement, step e) of separating the stream rich in aromatics is carried out in a divided-wall distillation column.

A divided-wall column is an item of distillation equipment well known to those skilled in the art in which an internal wall leaktight to fluids and arranged vertically separates a portion of the column into two distinct zones. A divided-wall column thus generally consists of a lower common portion in which the separation stages are not divided by the internal wall, a divided portion in which the separation stages are divided by the internal wall, and an upper common portion in which the separation stages are not divided by the internal wall.

The divided-wall column comprises a total of from 70 to 130 theoretical stages, preferably from 80 to 120 theoretical stages, very preferably from 90 to 110 theoretical stages. The internal wall is preferably centred, that is to say it partitions the column in the length in which it is present into two parts of equal volume. When the number of theoretical stages on either side of the internal wall is different, for example because of the use of different types of distributor plates or different packing, the total number of theoretical stages of the column is understood as meaning the sum of the theoretical stages of the common parts and of the larger number of stages between the two divided parts. The column is operated at a pressure of less than or equal to 0.25 bara at the head of the column so as to maintain the temperature at the column bottom at a value of less than or equal to 120° C.

The stream rich in aromatics from step c) of the process is supplied on one side of the internal wall at a stage ranging from the 10th to the 20th theoretical stage, preferably ranging from the 12th to the 18th theoretical stage and very preferably to the 15th theoretical stage, the stages being numbered from the top downwards.

In a first variant of this arrangement, the lower common portion of the divided-wall column comprises from 8 to 12 theoretical stages and the upper common portion comprises from 8 to 12 theoretical stages. The stream comprising predominantly styrene is withdrawn in the portion on the opposite side of the internal wall to the portion in which the stream rich in aromatics is injected. The withdrawal is carried out at a stage close to the upper portion of the divided portion, preferably at one of the 5 upper stages of the divided portion, preferably at one of the 3 upper stages of the divided portion, more preferably at one of the two upper stages of the divided portion and very preferably at the first stage of the divided portion, counting the stages from the top.

The stream comprising predominantly ethylbenzene is withdrawn at the head of the column and the stream of heavy compounds at the column bottom.

The reflux ratio at the condenser of this column, corresponding to the reflux mass flow supplied at the head of the column to the mass flow of the stream comprising predominantly ethylbenzene, is preferably between 60 and 300. This parameter varies greatly, depending on the ethylbenzene content of the stream rich in aromatics. The lower the content of ethylbenzene in the stream rich in aromatics, the higher the reflux ratio at the condenser of the column.

The reflux ratio at the reboiler of this column, corresponding to the reflux mass flow supplied at the column bottom to the mass flow of the stream of heavy compounds, is preferably between 50 and 200, this ratio being influenced greatly by the content of compounds such as cumene and α-methylstyrene.

In a second variant of this arrangement, the divided-wall column does not comprise an upper common portion, that is to say the wall extends up to the top of the divided-wall column.

In this variant, the lower common portion comprises from 2 to 12 theoretical stages, preferably from 2 to 10 theoretical stages and very preferably from 2 to 4 theoretical stages.

In this variant, the divided-wall column preferably comprises a total of from 60 to 80 theoretical stages.

In this variant, the stream comprising predominantly styrene is withdrawn in the portion on the opposite side of the internal wall to the portion in which the stream rich in aromatics is injected. The withdrawal is carried out at the head of the column.

The stream comprising predominantly ethylbenzene is withdrawn at the head of the column in the same portion as the portion in which the stream rich in aromatics is injected.

The reflux ratio at the condenser of this column for the divided portion located on the side of the supply of the stream rich in aromatics, corresponding to the reflux mass flow supplied at the head of the column in this portion to the mass flow of the stream comprising predominantly ethylbenzene, is preferably between 60 and 300. This parameter varies greatly, depending on the ethylbenzene content of the stream rich in aromatics. The lower the content of ethylbenzene in the stream rich in aromatics, the higher the reflux ratio at the condenser of the column.

The reflux ratio at the condenser of this column for the divided portion located on the side of the withdrawal of the stream comprising predominantly styrene, corresponding to the reflux mass flow supplied at the head of the column to the mass flow of the stream comprising predominantly styrene, is preferably between 1 and 10.

The reflux ratio at the reboiler of this column, corresponding to the reflux mass flow supplied at the column bottom to the mass flow of the stream of heavy compounds, is preferably between 60 and 200, this ratio being influenced greatly by the content of compounds such as cumene and α-methylstyrene.

In order to limit the risk of polymerization still further, an inhibitor of the polymerization of styrene to give polystyrene, such as 2,2,6,6-tetramethyl-4-oxopiperidinooxy, may preferably be supplied to the column(s) employed in separation step c), preferably at the head of the column(s) employed in separation step c).

The stream comprising predominantly ethylbenzene from step e) is preferably separated into at least a stream comprising predominantly toluene, a stream that comprises ethylbenzene and a stream that comprises styrene.

The stream of heavy compounds from step e) is preferably separated into at least a stream comprising styrene, a stream that comprises AMS and a stream that comprises oligomers.

### DESCRIPTION OF THE FIGURES

For each of the following figures, identical numbers and letters correspond to similar streams and operations.

FIG. 1 illustrates schematically a prior art process for depolymerizing a polystyrene feedstock.

A polystyrene feedstock (1) supplies an extrusion section (A) in which it is gradually heated so as to melt said feedstock, the liquid fraction (2) being separated from the solid fraction (3). The liquid fraction (2) is sent to a mixing tank (B), where it is mixed with the liquid fraction (7) from the liquid-solid separator (C).

The mixing tank (B) supplies the pyrolysis reactor (D), which produces a first gaseous pyrolysis stream (5) and a first liquid pyrolysis stream (6), the latter being separated in the liquid-solid separator (C) into any solid fraction (8) that is present and a liquid fraction (7).

The first gaseous pyrolysis stream (5) supplies a first distillation column (E) that produces at the head a stream rich in light compounds (9), at the bottom a stream rich in oligomers (11), and as a sidestream withdrawal a stream rich in aromatics (10).

The latter supplies a distillation column (F) in which it is separated into a stream comprising predominantly ethylbenzene (12) and a styrene raffinate (13) that supplies a distillation column (G) separating it into a stream comprising predominantly styrene (14) and a stream of heavy compounds (15).

FIG. 2 illustrates schematically a possible configuration for a process for depolymerizing a polystyrene feedstock according to the invention.

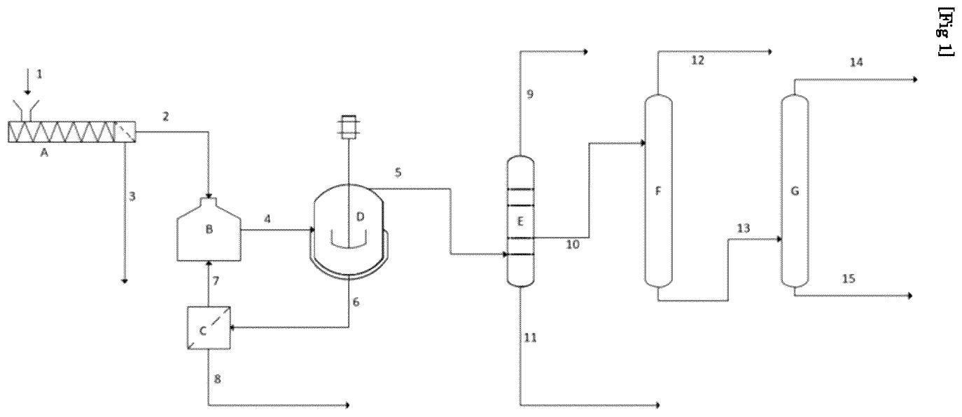

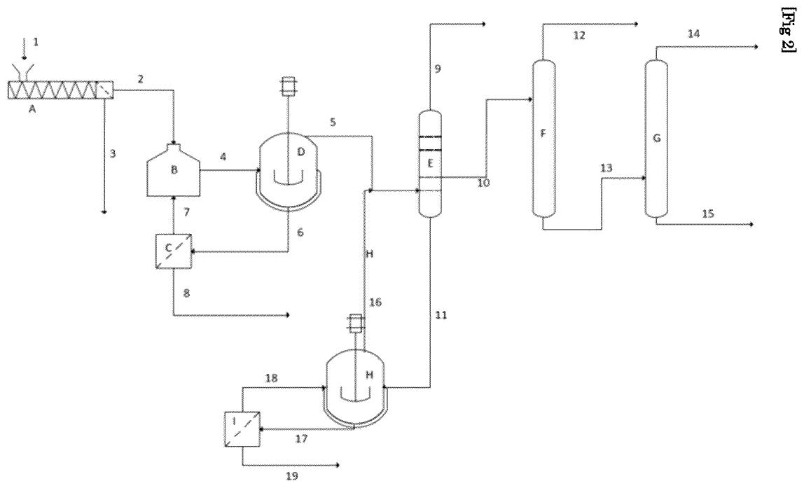

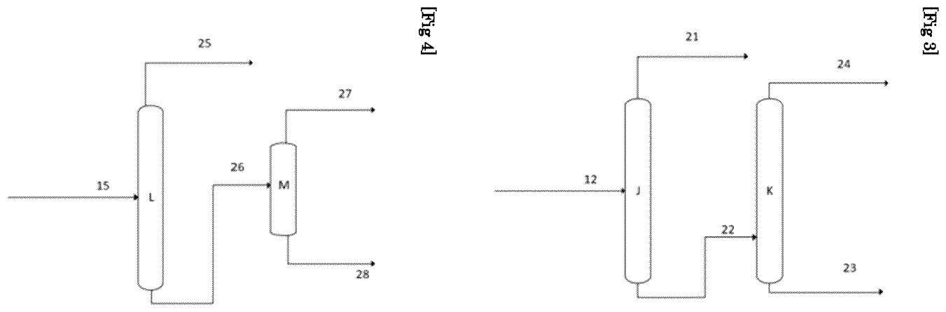

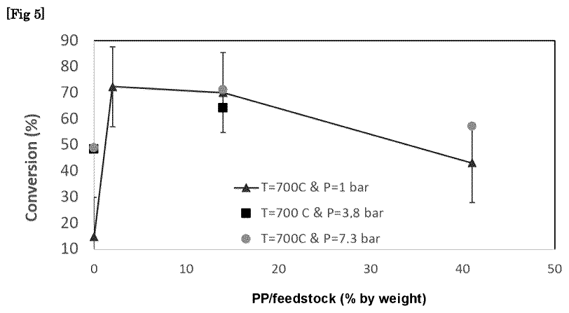

The elements that are functionally identical to those of FIG. 1 are numbered in the same way, although this does not mean that they have the same dimensions or are operated under the same conditions.

In this arrangement, the stream rich in oligomers (11) supplies a pyrolysis reactor (H) that produces a second gaseous pyrolysis stream (16), the latter being mixed with the first gaseous pyrolysis stream (5) prior to being supplied to the distillation column (E). The pyrolysis reactor (H) also produces a second liquid pyrolysis stream (17). The second liquid pyrolysis stream (17) is separated in the liquid-solid separator (I) into any solid fraction (19) that is present and a liquid fraction (18) that is supplied to the pyrolysis reactor (H).

In this arrangement, which is not shown in this diagram, it is possible to adjust the functioning of the pyrolysis reactor (H) by injecting an inert gas in a mixture with the oligomer-rich stream (11) or to control the hydrogen partial pressure of the pyrolysis reactor (H) through the presence of a hydrogen donor in the polystyrene feedstock (1) and/or in the stream rich in oligomers (11).

FIG. 3 illustrates schematically an arrangement in which a stream comprising predominantly ethylbenzene (12) supplies a distillation column (J) comprising preferably from 20 to 40 theoretical stages, in this case 30, substantially in the middle thereof, in this case at theoretical plate 30. This column (J) produces a stream comprising predominantly toluene (21) and a raffinate (22). The latter supplies a distillation column (K) comprising preferably from 50 to 80 theoretical stages, in this case 65, in the upper portion of its lower half, in this case at theoretical plate 30. This column (K) produces a stream (24) that comprises ethylbenzene and a stream (23) that comprises styrene. This arrangement makes it possible to maximize the recovery of these constituents.

FIG. 4 illustrates schematically an arrangement in which a stream of heavy compounds (15) is treated in order to maximize the recovery of styrene and of alpha-methylstyrene (AMS). The stream of heavy compounds (15) supplies a distillation column (L) comprising preferably from 15 to 25 theoretical stages, in this case 20, substantially in the middle thereof, in this case at theoretical plate 10. This column (L) produces a stream (25) comprising styrene and a raffinate (26). The latter supplies a distillation column (M) comprising from 1 to 3 theoretical stages, in this case a flash comprising a single theoretical stage, substantially in the middle thereof when the column comprises a plurality of theoretical stages. This column (M) produces a stream (27) that comprises AMS and a stream (28) that comprises oligomers.

FIG. 5 is a representation of the conversion of oligomers as a function of the mass fraction of hydrogen donor, in this case polypropylene added to the polystyrene feedstock, calculated as the ratio of the flow rate of the polypropylene to the sum of the flow rate of the polypropylene and the flow rate of the polystyrene in the polystyrene feedstock, at a temperature of 700° C. for various operating pressures in the pyrolysis step.

### EXAMPLES

### Example 1

This example illustrates the depolymerization of polystyrene according to the scheme shown in FIG. 1.

A polystyrene feedstock (1), in this case polystyrene, supplies an extruder (A) in which it is brought to a temperature of 250° C. After being mixed with the liquid fraction from the pyrolysis reactor (7), the liquid fraction of the feedstock (2) supplies a pyrolysis reactor (D), in this case a microwave pyrolysis reactor, operated at a temperature of 340° C. and at a pressure of 1.1 bar. The first gaseous pyrolysis stream (5) is separated into a stream rich in light compounds (9), a stream rich in oligomers (11), a stream comprising predominantly ethylbenzene (12), a stream comprising predominantly styrene (14) and a stream of heavy compounds (15).

The characteristics of the distillation columns E, F and G are presented in Table 1.

The amounts of styrene, toluene, alpha-methylstyrene and other aromatics produced are indicated in Table 2.

### Example 2

This example illustrates the depolymerization of the polystyrene according to the invention, according to the scheme shown in FIG. 2, the streams (12) and (15) being treated according to the schemes presented in FIGS. 3 and 4.

The pyrolysis reactor (D), in this case a microwave pyrolysis reactor, is operated at a temperature of 340° C. and a pressure of 1.1 bar. The second pyrolysis reactor (H), in this case a microwave pyrolysis reactor, is operated at a temperature of 700° C. and a pressure of 3.7 bar.

The characteristics of the distillation columns E, F, G, J, K, L and M are presented in Table 1.

Table 2 shows the results in terms of overall production in three situations:
Example 2a: the polystyrene feedstock comprises 2% by weight of polypropylene
Example 2b: the polystyrene feedstock comprises 14% by weight of polypropylene

The polypropylene acts as a hydrogen donor. It is observed that the presence of a hydrogen donor in the feedstock makes it possible to improve the overall yield of aromatic compounds of interest, and of styrene in particular. However, if present in too high a proportion, this will adversely affect the overall yield.

TABLE 1

| Column | Unit | E | F | G | J | K | L | M |
|---|---|---|---|---|---|---|---|---|
| Number of theoretical | — | 10 | 60 | 50 | 30 | 65 | 20 | 1 |
| stages | | | | | | | | |
| Supply stage | — | 8 | 20 | 25 | 15 | 30 | 10 | 1 |
| Condenser pressure | mbar | 1013 | 77 | 47 | 200 | 200 | 85 | 85 |
| Condenser temperature | ° C. | 2 | 35 | 35 | 61 | 85 | 72 | 88 |
| Reboiler temperature | ° C. | 224 | 80 | 80 | 87 | 92 | 88 | 95 |

TABLE 2

| | | Example 2a | Example 2b |
|---|---|---|---|
| | | PP/feedstock | PP/feedstock |
| Per 100 kg/h of pure PS | Example 1 | 2% by weight | 14% by weight |
| Styrene prod. | 58.6 | 60.0 | 60.6 |
| Toluene prod. | 1.8 | 5.3 | 5.1 |
| AMS prod. | 9.2 | 9.8 | 10.1 |
| Other aromatics prod. | 2.6 | 5.3 | 5.2 |
| Total aromatics | 72.1 | 80.3 | 81.0 |
| Non-condensable prod. | 8.4 | 13.6 | 12.9 |
| Light oligomers prod. | 16.8 | 0.0 | 0.0 |
| Solid residues prod. | 2.7 | 6.1 | 6.1 |

1.-14. (canceled)

15. A process for depolymerizing a polystyrene feedstock, comprising at least the following steps:
(a) a step of preparing the polystyrene feedstock so as to bring about melting of plastic compounds;
(b) a step of performing a first pyrolysis of the feedstock resulting from step (a) involving a pyrolysis section and producing at least a first gaseous pyrolysis stream and a first liquid pyrolysis stream;
(c) a separating step supplied at least by the first gaseous pyrolysis stream from step (b) and a second gaseous pyrolysis stream from step (d) and producing at least a stream rich in light compounds, a stream rich in oligomers, and a stream rich in aromatics;
(d) a step of performing a second pyrolysis involving a pyrolysis section supplied at least by the stream rich in oligomers from step (c) and producing at least the second gaseous pyrolysis stream and a second liquid pyrolysis stream; and
(e) a step of separating the stream rich in aromatics from step (c) into at least a stream comprising predominantly ethylbenzene, a stream comprising predominantly styrene and a stream of heavy compounds,
wherein a hydrogen partial pressure and a residence time of gas phase are controlled in the pyrolysis section in step (d).

16. The process according to claim 15, wherein the hydrogen partial pressure in the pyrolysis section in step (d) is controlled by presence of a hydrogen donor in the polystyrene feedstock and/or in the supply to the pyrolysis section in step (d) and/or by supplying an inert gas to the pyrolysis section in step (d).

17. The process according to claim 16, wherein the hydrogen partial pressure in the pyrolysis section in step (d) is controlled by the presence of a hydrogen donor selected from polyolefins, paraffin waxes and mixtures thereof in the polystyrene feedstock and/or in the stream rich in oligomers from step (c).

18. The process according to claim 17, wherein the hydrogen partial pressure in the pyrolysis section in step (d) is controlled by the presence of a hydrogen donor in the styrene feedstock, the hydrogen donor being polypropylene and a polypropylene to polystyrene weight ratio in the polystyrene feedstock being adjusted to between 1% by weight and 15% by weight.

19. The process according to claim 15, wherein the hydrogen partial pressure in the pyrolysis section in step (d) is controlled by the presence of a hydrogen donor selected from polyolefins, paraffin waxes, dihydrogen and mixtures thereof in the supply to the pyrolysis section in step (d).

20. The process according to claim 15, wherein the second pyrolysis step (d) is operated at a temperature ranging from 400° C. to 900° C. and at a pressure ranging from 1.0 bar to 7.5 bar.

21. The process according to claim 15, wherein the second pyrolysis step (d) is operated with a gas-phase residence time ranging from 10 s to 30 s.

22. The process according to claim 21, wherein the residence time of the gas phase is controlled by supplying an inert gas to the pyrolysis section in step (d).

23. The process according to claim 15, wherein the first pyrolysis step (b) and/or the second pyrolysis step (d) employs a microwave pyrolysis section.

24. The process according to claim 15, wherein the separation step (c) employs a distillation column supplied at a bottom of the column with the first gaseous pyrolysis stream from step (b) and the second gaseous pyrolysis stream from step (d) and producing at a head of the column a stream rich in light compounds and at the bottom a stream rich in oligomers and, through a sidestream withdrawal, a stream rich in aromatics, the column having as sole heat input the first gaseous pyrolysis stream from step (b) and the second gaseous pyrolysis stream from step (d).

25. The process according to claim 15, wherein step (e) of separating the stream rich in aromatics includes a first separation section supplied with the stream rich in aromatics from step (c), which makes it possible to separate a stream comprising predominantly ethylbenzene and a styrene raffinate, and a second separation section supplied with the styrene raffinate from the first separation section and producing a stream comprising predominantly styrene and a stream of heavy compounds.

26. The process according to claim 15, wherein step (e) of separating the stream rich in aromatics is carried out in a divided-wall distillation column.

27. The process according to claim 15, wherein the stream comprising predominantly ethylbenzene from step (e) is separated into at least a stream comprising predominantly toluene, a stream that comprises ethylbenzene and a stream that comprises styrene.

28. The process according to claim 15, wherein the stream of heavy compounds from step (e) is separated into at least a stream comprising styrene, a stream that comprises alpha-methylstyrene and a stream that comprises oligomers.
